(19) United States
(12) Patent Application Publication
(10) Pub. No.: US 2009/0317464 A1
(43) Pub. Date: Dec. 24, 2009

(54) METHODS AND COMPOSITIONS FOR PRODUCING ANTI-ANDROGENIC EFFECTS

(75) Inventors: Amarjit Singh, Mumbai (IN); Sarabjit Singh, Mumbai (IN); Shivanand Puthli, Mumbai (IN)

Correspondence Address: LADAS & PARRY LLP, 26 WEST 61ST STREET, NEW YORK, NY, 10023, US

(73) Assignee: PANACEA BIOTEC LTD., Mumbai, Maharastra (IN)

(21) Appl. No.: 12/305,025

(22) PCT Filed: Jul. 5, 2007
(86) PCT No.: PCT/IN2007/000274

§ 371 Date: Dec. 16, 2008

(30) Foreign Application Priority Data
Jul. 7, 2006 (IN) 1074/MUM/2006

(51) Int. Cl. A61K 9/24; A61K 31/167; A61K 9/20; A61P 5/28

(52) U.S. Cl. 424/472; 514/618; 424/464

(57) ABSTRACT

The invention provides a method and a composition for producing an anti-androgenic effect in a mammal. The method comprises administering a modified release pharmaceutical composition of bicalutamide to a mammal, with a reduced dosing frequency, for improved patient convenience and compliance. The composition of the invention also provides for a higher bioavailability and improved pharmacokinetic profile as compared to a conventional bicalutamide composition.

### FIELD OF THE INVENTION

This invention belongs to the field of pharmaceutical science. It relates to methods and compositions of bicalutamide, a non-steroidal anti-androgen compound.

### BACKGROUND OF THE INVENTION

Androgens are natural or synthetic compounds, usually steroid hormones, which stimulate and control the development and maintenance of masculine characteristics by binding to androgen receptors. Being anabolic steroids, they are known to stimulate growth of certain tissues like the prostate and other peripheral tissues, including primary or metastatic prostate tumor cells. Androgens like testosterone and dihydrotestosterone exert their influence on cell functions by binding to androgen receptors. The high response rate to first line ‘Androgen deprivation therapy’ and the presence of androgen receptors in both primary and metastatic prostate tumor cells support the idea that the receptor is an important mediator of prostate cancer development and growth.

Prostatic carcinomas are androgen dependent; hence various treatment strategies focus on negating the role of androgens (i.e., testosterone and dihydrotestosterone) in prostate tumor growth. These treatment strategies include use of luteinizing hormone-releasing hormone (“LHRH”) analogues to suppress testicular androgen production (chemical castration) or orchiectomy (surgical castration) to eliminate androgen production.

In recent years, a class of drugs, known as anti-androgens, has widely been used for the treatment of prostate cancer. The biologic activity of androgens is mediated through the formation of a non-covalent androgen receptor-steroid complex. Anti-androgens inhibit formation of this complex and, thus, negate the role of endogenous steroids in androgen-dependent growth of the prostate.

Combinations of the above mentioned strategies have also been studied in a number of cases to evaluate the relative efficacies and survival benefits. A combination of anti-androgen drugs with castration (either chemical or surgical), not only prevents the action of testosterone produced by the testes but also the small, but important amount which is produced by other glands i.e. the adrenal glands. Such therapy is sometimes called Complete or Maximal Androgen Blockade (CAB or MAB). In advanced disease a combination of an LHRH analogue (e.g. goserelin, leuprolide) (generally administered as long acting injectable implants or depots) and a non-steroidal anti-androgen (e.g. bicalutamide, given as oral tablets) can be used. Clinical trials identify that men treated with such combination therapy may live longer than those treated with an LHRH analogue alone. Combination therapy may sometimes also be used prior to surgery or radiotherapy (neoadjuvant) to reduce the size of the tumour.

Bicalutamide, an acylanilide, is a non-steroidal anti-androgen, is a ‘second generation’ anti-androgen, which has more advantageous properties than the other known anti-androgens, like Flutamide or Nilutamide. (For example, refer to Schellhammer P F et al., Urology, 50: 330-6, (1997); and to McLeod D G, Oncologist, 2:18-27, (1997).) The commercially available product by AstraZeneca is CASODEX™ and its chemical formula is 4-cyano-3-trifluoromethyl-N-(3-p-fluorophenylsulphony 1-2-hydroxy-2-methylpropionyl) aniline. The compound has been disclosed in patents like U.S. Pat. No. 4,636,505 and European Patent No. 100172. Its anti-androgenic activity resides almost exclusively in (R)-bicalutamide, with little, if any, activity in (S)-bicalutamide. It is used at a dosage of 50 mg once daily in combination with a luteinising hormone-releasing hormone analogue or surgical castration for the treatment of advanced prostate cancer. It is also given at a dosage of 150 mg once daily as monotherapy for the treatment of early (localized or locally advanced) non-metastatic prostate cancer. It has a long half life of approximately 5.8 days and time to peak plasma concentration of about 31 hours.

Reports on bicalutamide detail that after daily administration of bicalutamide to patients the relationship between steady state concentration (Css) and dose is linear only between 10 and 50 mg/day, it shows departure from linearity at doses between 100 and 200 mg per day and increased departure from linearity at 300 mg/day. Above 300 mg, especially when studied at 450 and 600 mg dose, no further increase in Css is observed. This non-linearity and plateau in Css at higher dose has been attributed to decreased drug absorption and saturable transport across GI tract. Refer to Tyrell C J et al., Eur Urol 33:39-53, (1998).

Prostate cancer most often advances very slowly and many patients often have no symptoms, particularly in the early stages. Most men with prostate cancer will live with their disease for many years. Hence it is important to consider their choice when faced with the androgen deprivation therapies mentioned above. For example, refer to Nyman, et al., BJU International, 96: 1014-1018, (2005), where a Swedish study in 150 patients was conducted to investigate their choice amongst the three androgen deprivation therapies: bicalutamide, GnRH analogue and orchiectomy. Most men preferred oral tablets of anti-androgen bicalutamide, but they also indicated dissatisfaction; one of the main reasons for which was the need to take the drug daily. Bicalutamide is often used for months to years together. Hence, in such situations, it becomes difficult to take medications daily and there is need for a therapy which reduces the dosing frequency, thereby increasing patient comfort and compliance.

United States Application No. 2004067257 discloses solid dispersions of bicalutamide with enteric polymers having a pKa of 3 to 6 for increasing the bioavailability of the drug for reducing inter-patient variability in plasma concentrations of bicalutamide; or for treating and/or reducing the risk of prostate cancer in a patient. The patient has to take the pharmaceutical dose daily.

International Publication WO2006069098 discloses nanoparticulate bicalutamide formulations, having bicalutamide particles with an effective average particle size of less than about 2000 nm and containing surface stabilizers, which target an increase in the bioavailability of the drug. The publication teaches compositions that can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like. The invention relates to increasing the solubility and bioavailability of bicalutamide.

International Publication WO2006076533 discloses controlled release compositions of acylanilide particles, particularly bicalutamide, for use in combination therapy and which are purported to deliver the active ingredient in a pulsed or bi-modal manner on oral administration. The compositions are multiparticulate where the subsequent pulses are obtained by coating the population of particles. No details of formulation or any examples are given. The compositions are meant for once daily administration.

International Publication WO2006090129 relates to a method for treating metastatic prostate cancer by administering bicalutamide formulations which deliver mean steady state plasma levels of R-bicalutamide of at least 40 ug/ml. Examples given in the application are mostly related to solid dispersions and R-bicalutamide formulations, meant to be given daily.

Some attempts have been made earlier to improve the absorption and bioavailability of bicalutamide at higher doses but still there is a need to develop and provide a composition of bicalutamide that can overcome the saturable absorption and provide a linear relationship between steady state concentration and dosage over a wide dose range of from about 50 to about 600 mg.

It is also desirable to have a method for producing an anti-androgenic effect in a mammal by providing bicalutamide with a reduced dosing frequency and maintaining its therapeutically effective levels for a prolonged period of time. For such a method, although the dose administered during a dosing interval may be same or lesser as compared to the total dose given during the interval by conventional once-daily therapy, the method utilizes a higher one-time dose as compared to a conventional once-daily dose. This dose may be 150 mg or higher i.e. higher than the once-daily unit dose. However, as is known, for high doses, bicalutamide exhibits non-linear kinetics, possibly due to its saturable absorption. (Also, refer to Kolvenbag et al., Prostate, 34:61-72, (1998). Hence, inclusion of such doses may result in incomplete absorption, leading to sub-optimal drug levels in the plasma. Furthermore, at higher doses, high inter-patient variability in the pharmacokinetics of bicalutamide has been reported.

Therefore, there is a need in the art for a method for producing an anti-androgenic effect in a mammal in need thereof which solves the above and other problems.

### BRIEF DESCRIPTION OF THE INVENTION

The inventors have now found that contrary to the expected saturation in bicalutamide absorption, it is possible to administer bicalutamide at higher doses such that therapeutically effective plasma levels of the drug are maintained in the body for a prolonged period of time.

The method and composition of the present invention provide for maintenance of therapeutically effective plasma level of bicalutamide for a prolonged period of time, and hence allow for a reduced dosing frequency.

An aspect of the present invention relates to a method for producing an anti-androgenic effect in a mammal in need thereof by oral administration of a pharmaceutically effective amount of bicalutamide with a reduced dosing frequency, such as thrice-a-week dosing, twice-a-week dosing and once-a-week dosing.

Another aspect of the present invention relates to a method for producing an anti-androgenic effect in a mammal by orally administering to the mammal a modified release pharmaceutical composition of bicalutamide in a once-a-week dosing schedule.

The present invention provides a once-a-week composition containing bicalutamide for producing an anti androgenic effect in a mammal.

An aspect of the present invention also relates to a pharmaceutically effective amount of bicalutamide which ranges from about 50 mg to about 1000 mg and which will be effective for the method of the invention.

Another aspect of the present invention relates to a modified release pharmaceutical composition of bicalutamide which exhibits higher bioavailability and improved pharmacokinetic profile of bicalutamide. The composition is an oral pulsed release composition wherein part of the dose of bicalutamide is released substantially immediately upon administration and the remaining part of the dose is released after a pre-determined time interval. The immediate release portion is formulated in the form of at least one uncoated or film coated tablet and the delayed release portion is formulated in the form of at least one tablet coated with delayed release polymers.

### DESCRIPTION OF THE DRAWINGS

FIG. 1 shows the predicted plasma concentration time profiles of the Test and Reference bicalutamide compositions at steady state levels, in comparison with the predicted reference of 50 mg bicalutamide administered daily.

FIG. 2 shows the dissolution profile of an illustrative composition of bicalutamide (“Test”) from Example 3.

### DESCRIPTION OF THE INVENTION

The present invention relates to a method, preferably an oral method, for producing an anti-androgenic effect in a mammal in need thereof.

The method is provided by orally administering to a mammal, a modified release pharmaceutical composition of bicalutamide, which is administered in a thrice-a-week, twice-a-week or once-a-week dosing schedule.

Preferably, the modified release pharmaceutical composition of bicalutamide is administered in a once-a-week dosing schedule.

The method of the invention provides for prolonged maintenance of therapeutically effective plasma levels of bicalutamide in the body. The dosing frequency is reduced by providing a modified release pharmaceutical composition that reduces bicalutamide losses due to saturation of absorption mechanisms. Moreover, the method of the present invention provides for improved patient compliance since the inconvenience of daily dosing is avoided. The method may also provide for reduced inter-patient variability.

The term ‘bicalutamide’ as used herein contemplates all forms of bicalutamide, such as the free base, any pharmaceutically acceptable salts, esters, solvates thereof as well as the racemate, the individual isomers R-bicalutamide and S-bicalutamide and their pharmaceutically acceptable salts, esters and solvates. Accordingly, bicalutamide is used in the pharmaceutical composition of the present invention in the range of amounts equivalent to about 50 mg to about 1000 mg of bicalutamide.

‘Thrice-a-week’ dosing schedule as used herein, means that a dose of bicalutamide is administered three times in a week i.e. three times during a seven day period, preferably on the same three days of each weekly period.

‘Twice-a-week’ dosing schedule as used herein, means that a dose of bicalutamide is administered two times in a week i.e. two times during a seven day period, preferably on the same two days of each weekly period.

The term ‘once-a-week’ dosing schedule as used herein means that a dose of bicalutamide is administered once in a week i.e. one time during a seven day period, preferably on the same day of each week.

The term ‘pharmaceutically effective amount’ of bicalutamide as used herein indicates an amount of bicalutamide necessary to be administered to a mammal to achieve a desired therapeutic result, especially an anti-androgenic effect. The dose utilized for the above purpose will generally be from about 50 mg to about 1000 mg.

‘Reduced dosing frequency’ as used herein means that the frequency of administration of bicalutamide to a mammal is less than once in a day. Thus, for example, the frequency may be thrice in a week, twice in a week or once in a week.

The method of the present invention is generally applicable to a mammal in need of anti-androgen therapy. Preferably the mammal is a human patient, more preferably male, and particularly a male who has been identified as suffering from proliferative disorders, such as prostate cancer, benign prostate hyperplasia or possibly any other genito-urinary cancers.

An aspect of the method of the present invention contemplates administering the bicalutamide composition of the present invention alone as well as in combination with other pharmaceutical agents such as anti-estrogens, for example tamoxifen; aromatase inhibitors, for example anastrazole; LH-RH analogues, for example goserelin; cytotoxic agents, such as cyclophosphamide; and any other suitable agents. The composition may also be administered as an adjunct with orchiectomy.

Another aspect of the method of the present invention contemplates administration of one or more loading doses of bicalutamide, followed by administration of the composition of the invention as a maintenance therapy. This will allow for rapid attainment of steady state levels followed by their maintenance over the period of therapy.

The composition of the invention is administered as a unit dosage and the unit dose may range from about 50 mg to about 1000 mg of bicalutamide. Preferably it varies from about 150 mg to about 600 mg and more preferably from about 150 mg to about 400 mg of bicalutamide administered in a thrice-a-week, twice-a-week or once-a-week dosing schedule.

The modified release pharmaceutical composition of the present invention comprises of bicalutamide and suitable excipients. The composition may demonstrate any type of modified release profile which would allow for a reduced dosing frequency. Thus the modified release profile according to the invention may be a controlled release, sustained release, extended release, delayed release, pulsed release, dual release, timed release, site-specific release and others, including any combinations thereof.

In one embodiment of the invention, the composition is in the form of an oral delayed release product, wherein upon administration, the entire dose of bicalutamide is released after a pre-determined time interval, in the later part of the gastro-intestinal tract. The release may be either ‘all-at-once’ or in a sustained manner.

In an embodiment of the invention, the composition is in the form of an oral pulsed release product, wherein part of the dose of bicalutamide is released substantially immediately upon administration and the remaining part of the dose is released after a pre-determined time interval.

In another embodiment of the invention, the composition is in the form of an oral pulsed release product, wherein part of the dose of bicalutamide is released substantially immediately upon administration and the remaining dose is released in a sustained manner after a pre-determined time interval.

The composition of the above embodiments may include various pharmaceutically acceptable excipients, for example, diluents such as microcrystalline cellulose, lactose, sucrose, calcium phosphates; disintegrants such as starch, cellulose derivatives, gums, crosslinked polymers and the like; binders such as starch, gelatin, sugars, cellulose derivatives, polyvinyl pyrrolidone and the like; lubricants such as talc, magnesium stearate, colloidal silicon dioxide, polyethylene glycol and mixtures thereof.

The composition can be formulated in the form of granules, tablets, layered tablets, mini-tablets and pellets or as capsules filled with powder, pellets, mini-tablets and/or tablets. The processes for the preparation of these compositions are well known to a person skilled in the art, and are included herein by reference.

In an embodiment, bicalutamide and suitable excipients are formulated together in the form of granules. The granules are compressed to form mini-tablets of bicalutamide. They may optionally be film-coated. These mini-tablets release bicalutamide substantially immediately on administration of the composition. The mini-tablets are then coated with suitable polymers which modify the release rate and time of bicalutamide. These polymers generally belong to categories such as celluloses, vinyl pyrrolidone polymers, alkylene oxide homopolymers, superdisintegrant polymers, acrylic acid polymers and gums of plant, animal, mineral or synthetic origin.

Preferably, the polymers delay the release of bicalutamide until a predetermined time interval. Preferred for the purpose are enteric polymers such as esters of cellulose and its derivatives, vinyl polymers and copolymers, pH-sensitive methacrylic acid copolymers, shellac etc. A few examples which can be used in the compositions of the invention are cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, etc.

In an embodiment, a hard gelatin capsule is filled with the coated mini-tablets, corresponding to the total dose of bicalutamide to be administered, to form the delayed release composition of the invention.

In a preferred embodiment, a hard gelatin capsule is filled with a combination of uncoated (or film coated) and coated mini-tablets. The number of uncoated and coated mini-tablets filled depends upon the amount of bicalutamide to be released immediately and after a predetermined period of time on administration. About 80 mg to about 600 mg of bicalutamide is included in the uncoated mini-tablets for immediate release and about 60 mg to about 400 mg of bicalutamide is included in the coated mini-tablets for delayed release.

In an alternative embodiment, the composition is formulated in the form of a single tablet, consisting of a core containing bicalutamide and coated with one or more polymers which modify the release rate of the drug.

In another alternative embodiment, the composition can be formulated as a matrix composition, wherein bicalutamide and the suitable polymer are mixed together to form a matrix which modifies the release of bicalutamide.

In still another embodiment, the composition is given as part of a kit comprising agents for combination therapy. Other agents which can be included as part of the kit are anti-estrogens, for example tamoxifen; aromatase inhibitors, for example anastrazole; LH-RH analogues, for example goserelin; cytotoxic agents, such as cyclophosphamide; and any other suitable agents.

In another aspect, the composition of the kit is administered daily, till a maximum of 4 weeks or till a steady state is achieved, as a loading dose, followed by weekly administration of the same composition as a maintenance dose.

The second aspect of the invention relates to a modified release pharmaceutical composition of bicalutamide. It has surprisingly been found that when the composition is formulated as a combination of immediate release and delayed release components in a specific manner, there is a substantial increase in the plasma levels of bicalutamide. It has also been surprisingly found that the area under the curve and the trough plasma levels for bicalutamide administered using the above composition were higher when compared with similar dose being given in conventional bicalutamide composition.

The composition is a solid oral composition, wherein the immediate release component is formulated as an uncoated tablet or film coated tablet which disperses to release bicalutamide substantially immediately upon oral administration. The delayed release component is formulated by coating the uncoated tablet with polymers which delay the release of bicalutamide for a predetermined time interval. The polymers are preferably enteric polymers selected from the group of esters of cellulose and its derivatives, vinyl polymers and copolymers and pH-sensitive methacrylic acid copolymers. Uncoated or film coated tablets and delayed release coated tablets are combined to form the modified release composition of the invention.

This composition can be used to obtain higher efficacy and better treatment of advanced, locally advanced, non-metastatic as well as metastatic prostate cancers. Dose and dosing regimen can be suitably selected depending upon the needs and disease state of the patients and depending upon whether it is given as part of combination therapy or as monotherapy.

An embodiment of this composition which demonstrates improved bioavailability when compared to conventional bicalutamide composition is a modified release pharmaceutical composition of bicalutamide wherein the composition is an oral pulsed release composition formulated as combination of immediate release and delayed release component comprising bicalutamide in an amount from about 20% to about 80% of the total dose in the immediate release component and in an amount from about 80% to about 20% of the total dose in the delayed release component.

In a specific embodiment, the modified release pharmaceutical composition when subjected to dissolution testing in USP apparatus Type I using pH change method with simulated gastric fluid of pH 1.2, buffer of pH 4.5 and simulated intestinal fluid of pH 6.8, it releases bicalutamide according to the following dissolution profile: between 20% and 65% of bicalutamide is released in simulated gastric fluid; between 40% and 75% is released in buffer of pH 4.5; and not less than 60% is released in simulated intestinal fluid. The dissolution media comprises of 1% sodium lauryl sulphate and the composition may be tested in simulated gastric fluid for about 2 hours, in buffer of pH 4.5 for about 2 hours and in simulated intestinal fluid for the remaining period of time.

### Clinical Study:

A study in a small group of volunteers was designed to evaluate the pharmacokinetics of a single dose administration of composition of the invention and compare it with the pharmacokinetics of a similar dose of Casodex® over a period of one week.

Study design: Open-label, randomized, fasted, single dose pharmacokinetic study using parallel study design. 12 healthy human volunteers were used (6 volunteers for each group). Test composition comprised 250 mg bicalutamide and was formulated to release part of the dose immediately upon administration and part of the dose after a predetermined time interval. Reference composition was five 50 mg Casodex® tablets administered together. Blood sampling was done pre-dose and after pre-determined time intervals over one week. Pharmacokinetic parameters assessed were Cmax, Tmax, AUCt and AUCinf.

### Results:

The pharmacokinetic data derived from the trial was as given in Table 1 below:

TABLE 1

| Product | N Obs | Parameter | N | Mean |
|---|---|---|---|---|
| R | 6 | Cmax (ng/ml) | 6 | 1774.43 |
| | | AUCt (ng · hr/ml) | 6 | 201978.96 |
| | | AUCinf (ng · hr/ml) | 6 | 325810.62 |
| T | 6 | Cmax (ng/ml) | 6 | 1928.17 |
| | | AUCt (ng · hr/ml) | 6 | 231661.88 |
| | | AUCinf (ng · hr/ml) | 6 | 489684.23 |

As seen from the table the AUCinf for the test product is about 1.5 times more than the AUCinf for the reference product indicating an improved absorption and bioavailability obtained from the composition of present invention. The increased absorption will be useful in faster buildup of steady state levels, in addition to reduced dosing/dosing frequency.

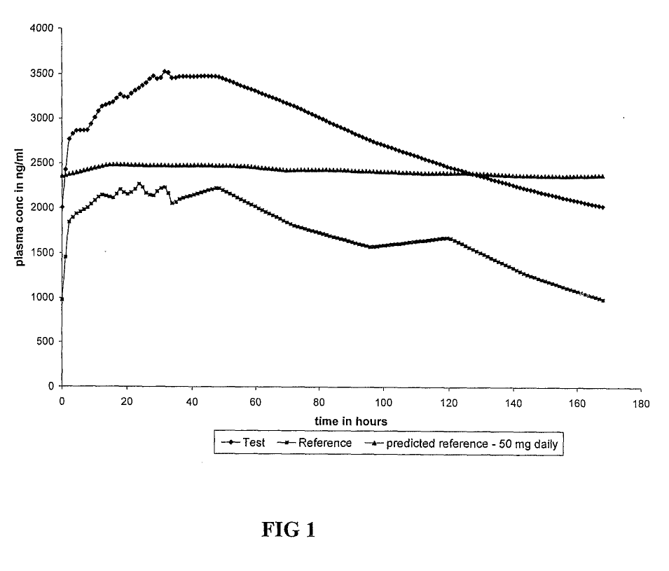

To predict and compare the kinetics of the Test and Reference compositions at steady state, the data from the trial was fed into the software Winnonlin®. The predicted plasma concentration time profiles of the Test and Reference compositions at steady state levels are shown in FIG. 1.

As can be seen from the figure, the method and the composition of the invention will result in an improved plasma profile of bicalutamide as compared to the Reference product. Also, the plasma level maintained by once-a-week administration over one week may be comparable or higher than the predicted plasma level of daily administration of 50 mg reference.

Steady state pharmacokinetic parameters were predicted for the Test and Reference products at 85^{th} day of dosing. The data is presented in the following Table 2.

TABLE 2

| | Least-Squares | | | 90% Confidence | |
|---|---|---|---|---|---|
| | Means | | | Interval | |
| Parameter | Test | Reference | Ratio | Lower | Upper |
| AUC 0-τ | 437704 | 320207 | 1.367 | 0.921 | 1.813 |
| (ng-hr/mL) | | | | | |
| Cmax | 3417 | 2625 | 1.302 | 0.972 | 1.631 |
| (ng/mL) | | | | | |
| Cmin | 1805 | 1148 | 1.572 | 0.907 | 2.236 |
| (ng/mL) | | | | | |
| Tmax | 32.8 | 27.7 | 1.184 | — | — |
| (hour) | | | | | |
| T½ | 131 | 105 | 1.251 | — | — |
| (hour) | | | | | |
| Fluctuation | 67.9 | 81.1 | 0.836 | — | — |
| (%) | | | | | |

As seen in the table 2 above for all the predicted steady state pharmacokinetic parameters, the test product has a higher value than the reference product, and the test product has less fluctuation compared to the reference product, indicating better absorption and bioavailability and an extended half life for the test product as well as a potential for reduced variability.

Various modifications of the method of the invention may be made without departing from the spirit or scope of the invention. The following non-limiting examples illustrate an embodiment of the invention and should not be construed to limit the scope of the invention.

### Example 1

TABLE 3
Formula (Core Tablet):

| Sr. No. | Ingredient | Qty (mg/tab) |
|---|---|---|
| 1 | Bicalutamide | 250.00 |
| 2 | Lactose monohydrate | 188.00 |
| 3 | Sodium starch glycolate | 35.00 |
| 4 | Polyvinyl pyrrolidone | 7.50 |
| 5 | Purified water | q.s. |
| 6 | Sodium starch glycolate | 17.50 |
| 7 | Magnesium stearate | 2.00 |

TABLE 4
Formula (Sub-Coat Composition):

| Sr. No. | Ingredients | Qty (gms/Kg) |
|---|---|---|
| 1 | Hydroxypropyl methylcellulose | 50.00 |
| 2 | Polyethylene Glycol (PEG 6000) | 7.50 |
| 3 | Methanol | 628.30 |
| 4 | Dichloromethane | 314.20 |

TABLE 5
Formula (Enteric Coat Composition):

| Sr. No. | Ingredients | Qty (gms/Kg) |
|---|---|---|
| 1 | Methacrylic acid co-polymer | 53.85 |
| | (Methacrylic Acid - Methyl | |
| | Methacrylate Copolymer (1:2)) | |
| 2 | Methacrylic acid co-polymer | 23.07 |
| | (Methacrylic Acid - Methyl | |
| | Methacrylate Copolymer (1:1)) | |
| 3 | Triethyl citrate | 15.38 |
| 4 | Acetone | 276.92 |
| 5 | Isopropyl alcohol | 553.07 |
| 6 | Purified water | 39.23 |
| 7 | Talc | 38.46 |

Bicalutamide, Sodium starch glycolate (intra-granular portion) and lactose monohydrate were sifted and blended in the amounts mentioned in the Table 2 above. Polyvinyl pyrrolidone dissolved in sufficient quantity of purified water was used to granulate the dry powder blend.

The blend was dried and sieved to obtain dry granules. The granules were then blended with the remaining amount of sodium starch glycolate and magnesium stearate. The lubricated blend was compressed into tablets using round punches.

The sub-coating solution was prepared by dissolving PEG 6000 and Hydroxypropyl Methyl Cellulose in the mixture of methanol and dichloromethane. The tablets were coated using the above solution, till a tablet weight gain of about 2-3% w/w was achieved.

The enteric coating dispersion was prepared by mixing acetone, isopropanol and purified water, and dispersing the methacryclic acid co-polymers, talc and triethyl citrate in them. The sub-coated tablets were coated with the above solution till a weight gain of 10-11% w/w was achieved.

### Example 2

TABLE 6
Formula (Core Tablet):

| Sr. No. | Ingredient | Qty (mg/tab) |
|---|---|---|
| 1 | Bicalutamide | 50.00 |
| 2 | Lactose monohydrate | 37.60 |
| 3 | Sodium starch glycolate | 7.00 |
| 4 | Polyvinyl pyrrolidone | 1.50 |
| 5 | Purified water | q.s. |
| 6 | Sodium starch glycolate | 3.50 |
| 7 | Magnesium stearate | 0.40 |

TABLE 7
Formula (Sub-Coat Composition):

| Sr. No. | Ingredients | Qty (gms/Kg) |
|---|---|---|
| 1 | Hydroxypropyl methylcellulose | 50.00 |
| 2 | Polyethylene Glycol (PEG 6000) | 7.50 |
| 3 | Methanol | 628.30 |
| 4 | Dichloromethane | 314.20 |

TABLE 8
Formula (Enteric Coat Composition):

| Sr. No. | Ingredients | Qty (gms/Kg) |
|---|---|---|
| 1 | Methacrylic acid co-polymer | 53.85 |
| | (Methacrylic Acid - Methyl | |
| | Methacrylate Copolymer (1:2)) | |
| 2 | Methacrylic acid co-polymer | 23.07 |
| | (Methacrylic Acid - Methyl | |
| | Methacrylate Copolymer (1:1)) | |
| 3 | Triethyl citrate | 15.38 |
| 4 | Acetone | 276.92 |
| 5 | Isopropyl alcohol | 553.07 |
| 6 | Purified water | 39.23 |
| 7 | Talc | 38.46 |

The process followed was the same as in Example 1. The tablets corresponding to a total dose of 250 mg bicalutamide (i.e. five coated tablets) were filled into empty hard gelatin capsule.

### Example 3

TABLE 9
Formula (Core Tablet):

| Sr. No. | Ingredient | Qty (mg/tab) |
|---|---|---|
| 1 | Bicalutamide (Micronized) | 50.00 |
| 2 | Lactose monohydrate | 61.00 |
| 3 | Sodium starch glycolate | 11.20 |
| 4 | Polyvinyl pyrrolidone | 2.40 |
| 5 | Purified water | q.s. |
| 6 | Sodium starch glycolate | 4.60 |
| 7 | Magnesium stearate | 0.60 |

TABLE 10
Formula (Sub-Coat Composition):

| Sr. No. | Ingredients | Qty (gms/Kg) |
|---|---|---|
| 1 | Opadry (Hypromellose coating powder) | 100 |
| 2 | Purified water q.s. | 900 |

TABLE 11
Formula (Enteric Coat Composition):

| Sr. No. | Ingredients | Qty (gms/Kg) |
|---|---|---|
| 1 | Methacrylic acid co-polymer | 53.850 |
| | (Methacrylic Acid - Methyl | |
| | Methacrylate Copolymer (1:2)) | |
| 2 | Methacrylic acid co-polymer | 23.078 |
| | (Methacrylic Acid - Methyl | |
| | Methacrylate Copolymer (1:1)) | |
| 3 | Triethyl citrate | 15.386 |
| 4 | Acetone | 276.920 |
| 5 | Isopropyl alcohol | 553.077 |
| 6 | Purified water | 39.230 |
| 7 | Talc | 38.460 |

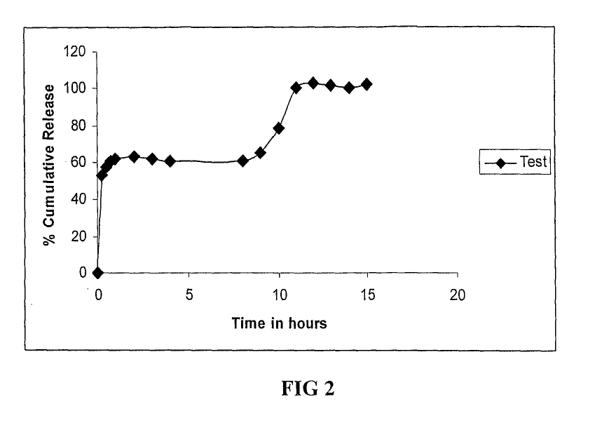

The process followed was the same as in Example 1. Three uncoated tablets, corresponding to a total dose of 150 mg and two coated tablets, corresponding to a total dose of 100 mg were filled into a hard gelatin capsule. The composition was subjected to a dissolution study using pH change method using buffers of pH 1.2, 4.5, and 6.8 with 1% Sodium Lauryl Sulphate (SLS). The study was carried out in USP apparatus Type 1. The dissolution profile obtained is indicated in FIG. 2.

### Example 4

TABLE 12
Formula (Core Tablet):

| Sr. No. | Ingredient | Qty (mg/tab) |
|---|---|---|
| 1 | Bicalutamide | 350.00 |
| 2 | Lactose monohydrate | 199.60 |
| 3 | Polyvinyl pyrrolidone | 18.00 |
| 4 | Sodium starch glycolate | 30.00 |
| 5 | Magnesium stearate | 2.40 |

TABLE 13
Formula (Sub-Coat Composition):

| Sr. No. | Ingredients | Qty (gms/Kg) |
|---|---|---|
| 1 | Hydroxypropyl methylcellulose | 50.00 |
| 2 | Polyethylene Glycol | 7.50 |
| 3 | Methanol | 628.30 |
| 4 | Dichloromethane | 314.20 |

TABLE 14
Formula (Enteric Coat Composition):

| Sr. No. | Ingredients | Qty (gms/Kg) |
|---|---|---|
| 1 | Methacrylic acid co-polymer | 80.00 |
| | (Methacrylic Acid - Methyl | |
| | Methacrylate Copolymer (1:2)) | |
| 2 | Methacrylic acid co-polymer | 6.4 |
| | (Methacrylic Acid - Methyl | |
| | Methacrylate Copolymer (1:1)) | |
| 3 | Triethyl citrate | 16.80 |
| 4 | Acetone | 297.90 |
| 5 | Isopropyl alcohol | 515.60 |
| 6 | Purified water | 42.40 |
| 7 | Talc | 43.20 |

The process of manufacturing was similar to that in Example 1.

### Example 5

TABLE 15
Formula (Core Tablet):

| Sr. No. | Ingredient | Qty (mg/tab) |
|---|---|---|
| 1 | Bicalutamide | 70.00 |
| 2 | Lactose monohydrate | 39.92 |
| 3 | Polyvinyl pyrrolidone | 3.60 |
| 4 | Sodium starch glycolate | 6.00 |
| 5 | Magnesium stearate | 0.48 |

TABLE 16
Formula (Sub-Coat Composition):

| Sr. No. | Ingredients | Qty (gms/Kg) |
|---|---|---|
| 1 | Hydroxypropyl methylcellulose | 50.00 |
| 2 | Polyethylene Glycol | 7.50 |
| 3 | Methanol | 628.30 |
| 4 | Dichloromethane | 314.20 |

TABLE 17
Formula (Enteric Coat Composition):

| Sr. No. | Ingredients | Qty (gms/Kg) |
|---|---|---|
| 1 | Methacrylic acid co-polymer | 80.00 |
| | (Methacrylic Acid - Methyl | |
| | Methacrylate Copolymer (1:2)) | |
| 2 | Methacrylic acid co-polymer | 6.4 |
| | (Methacrylic Acid - Methyl | |
| | Methacrylate Copolymer (1:1)) | |
| 3 | Triethyl citrate | 16.80 |
| 4 | Acetone | 297.90 |
| 5 | Isopropyl alcohol | 515.60 |
| 6 | Purified water | 42.40 |
| 7 | Talc | 43.20 |

The formula and process was similar to that in Example 1. The tablets corresponding to a total dose of 350 mg bicalutamide (i.e. five coated tablets) were filled into empty hard gelatin capsule shells.

### Example 6

TABLE 18
Formula (Core Tablet):

| Sr. No. | Ingredient | Qty (mg/tab) |
|---|---|---|
| 1 | Bicalutamide | 50.00 |
| 2 | Microcrystalline cellulose | 36.10 |
| 3 | Hydroxypropyl methylcellulose | 60.00 |
| 4 | Sodium starch glycolate | 3.50 |
| 5 | Magnesium stearate | 0.40 |

TABLE 19
Formula (Sub-Coat Composition):

| Sr. No. | Ingredients | Qty (gms/Kg) |
|---|---|---|
| 1 | Hydroxypropyl methylcellulose | 50.00 |
| 2 | Polyethylene Glycol | 7.50 |
| 3 | Methanol | 628.30 |
| 4 | Dichloromethane | 314.20 |

TABLE 20
Formula (Enteric Coat Composition):

| Sr. No. | Ingredients | Qty (gms/Kg) |
|---|---|---|
| 1 | Methacrylic acid co-polymer | 80.00 |
| | (Methacrylic Acid - Methyl | |
| | Methacrylate Copolymer (1:2)) | |
| 2 | Methacrylic acid co-polymer | 6.4 |
| | (Methacrylic Acid - Methyl | |
| | Methacrylate Copolymer (1:1)) | |
| 3 | Triethyl citrate | 16.80 |
| 4 | Acetone | 297.90 |
| 5 | Isopropyl alcohol | 515.60 |
| 6 | Purified water | 42.40 |
| 7 | Talc | 43.20 |

Bicalutamide, microcrystalline cellulose, and Hydroxypropyl methylcellulose were sifted and blended in the amounts mentioned in the Table 18 above. The mixture was further blended with sodium starch glycolate and lubricated with magnesium stearate. The lubricated blend was compressed into tablets on a rotary tablet compression machine. The tablets were sub coated and then enteric coated according to the manufacturing procedure mentioned in Example 1. The tablets corresponding to a total dose of 250 mg bicalutamide (i.e. five coated tablets) were filled into empty hard gelatin capsule shells.

### Example 7

TABLE 21
Formula (Immediate release Tablet):

| Sr. No. | Ingredient | Qty (mg/tab) |
|---|---|---|
| 1 | Bicalutamide | 100.00 |
| 2 | Lactose monohydrate | 75.20 |
| 3 | Sodium starch glycolate | 14.00 |
| 4 | Polyvinyl pyrrolidone | 3.00 |
| 5 | Purified water | q.s. |
| 6 | Sodium starch glycolate | 7.00 |
| 7 | Magnesium stearate | 0.80 |

TABLE 22
Formula (Sustained release Tablet):

| Sr. No. | Ingredient | Qty (mg/tab) |
|---|---|---|
| 1 | Bicalutamide | 250.00 |
| 2 | Microcrystalline cellulose | 180.50 |
| 3 | Hydroxypropyl methylcellulose | 300.00 |
| 4 | Sodium starch glycolate | 17.50 |
| 5 | Magnesium stearate | 2.00 |

Immediate release Tablet: Bicalutamide, Sodium starch glycolate (intra-granular portion) and lactose monohydrate were sifted and blended in the amounts mentioned in the Table 20 above. Polyvinyl pyrrolidone dissolved in sufficient quantity of purified water was used to granulate the dry powder blend. The blend was dried and sieved to obtain dry granules. The granules were then blended with the remaining amount of sodium starch glycolate and magnesium stearate. The lubricated blend was compressed into tablets using 6 mm round punches.

Sustained release Tablet: Bicalutamide, microcrystalline cellulose, and Hydroxypropyl methylcellulose were sifted and blended in the amounts mentioned in the Table 22 above. The mixture was further blended with sodium starch glycolate and lubricated with magnesium stearate. The lubricated blend was compressed into tablets on a rotary tablet compression machine. The tablets were sub coated and then enteric coated according to the manufacturing procedure mentioned in Example 1. Both the tablets of the example were filled into an empty hard gelatin capsule shell.

1. A method for producing an anti-androgenic effect in a mammal in need thereof comprising orally administering to the mammal a pharmaceutically effective amount of bicalutamide in a dosing schedule selected from the group of thrice-a-week dosing, twice-a-week dosing and once-a-week dosing.

2. The method of claim 1, comprising orally administering to the mammal a pharmaceutically effective amount of bicalutamide in a once-a-week dosing schedule.

3. The method of claim 1, wherein the pharmaceutically effective amount of bicalutamide ranges from about 150 mg to about 1000 mg.

4. A method for producing an anti-androgenic effect in a mammal in need thereof comprising orally administering to the mammal a modified release pharmaceutical composition of bicalutamide in a once-a-week dosing schedule.

5. The method of claim 4, wherein the modified release pharmaceutical composition of bicalutamide is administered as a monotherapy.

6. The method of claim 4, wherein the modified release pharmaceutical composition of bicalutamide is administered as part of a combination therapy.

7. A modified release pharmaceutical composition of bicalutamide for producing an anti-androgenic effect in a mammal in need thereof, wherein the composition is administered in a once-a-week dosing schedule.

8. The modified release pharmaceutical composition of claim 7 comprising bicalutamide in the range of from about 50 mg to about 1000 mg.

9. The modified release pharmaceutical composition of claim 8 comprising bicalutamide in the range of from about 150 mg to about 600 mg.

10. The modified release pharmaceutical composition of claim 7 formulated in the form of granules, tablets, layered tablets, mini-tablets, pellets or capsules.

11. The modified release pharmaceutical composition of claim 7 wherein the modified release is a controlled release, sustained release, extended release, delayed release, pulsed release, dual release, timed release or site-specific release.

12. The modified release pharmaceutical composition of claim 1 wherein the modified release is delayed release or pulsed release.

13. The modified release pharmaceutical composition of claim 7 wherein the composition is an oral pulsed release composition comprising at least one immediate release tablet and at least one tablet coated with delayed release polymers.

14. A modified release pharmaceutical composition of bicalutamide administered in a dosing schedule selected from the group of thrice-a-week dosing, twice-a-week dosing and once-a-week dosing.

15. A kit comprising a modified release pharmaceutical composition of bicalutamide for oral administration in a once-a-week dosing schedule.

16. The kit of claim 15, wherein the modified release pharmaceutical composition is administered once daily till a maximum of four weeks, as a loading dose, followed by weekly administration of the same composition as a maintenance dose.

17. The kit of claim 15, wherein the modified release pharmaceutical composition is administered once daily as a loading dose till a steady state plasma level of bicalutamide is achieved, followed by weekly administration of the same composition as a maintenance dose.

18. A modified release pharmaceutical composition of bicalutamide wherein the composition is an oral pulsed release composition formulated as combination of immediate release and delayed release component comprising bicalutamide in an amount from about 20% to about 80% of the total dose in the immediate release component and in an amount from about 80% to about 20% of the total dose in the delayed release component.

19. The modified release pharmaceutical composition of claim 18 wherein the oral pulsed release composition comprises of at least one immediate release tablet and at least one tablet coated with delayed release polymers, wherein the composition demonstrates improved bioavailability as compared to a conventional bicalutamide composition.

20. A modified release pharmaceutical composition of bicalutamide having improved bioavailability, wherein the composition comprises of one or more excipients or mixtures thereof, which extend the half life of bicalutamide.

21. The modified release pharmaceutical composition of claim 20, wherein the one or more excipients include polymers that modify the release rate and time of bicalutamide selected from the group of celluloses, vinyl pyrrolidone polymers, alkylene oxide homopolymers, superdisintegrant polymers, acrylic acid polymers and gums of plant, animal, mineral or synthetic origin.

22. The modified release pharmaceutical composition of claim 21, wherein the polymers are enteric polymers such as esters of cellulose and its derivatives, vinyl polymers and copolymers, pH-sensitive methacrylic acid copolymers, shellac and the like thereof.

23. A modified release pharmaceutical composition of bicalutamide which releases bicalutamide according to the following dissolution profile: between 20% and 65% of bicalutamide is released in simulated gastric fluid; between 40% and 75% is released in buffer of pH 4.5; and not less than 60% is released in simulated intestinal fluid; when tested in USP apparatus Type I using pH change method with simulated gastric fluid of pH 1.2, buffer of pH 4.5 and simulated intestinal fluid of pH 6.8 and wherein the dissolution media comprise of 1% sodium lauryl sulphate.

24. A modified release pharmaceutical composition of claim 23, wherein the dissolution testing is carried out in simulated gastric fluid for about 2 hours, in buffer of pH 4.5 for about 2 hours and in simulated intestinal fluid of pH 6.8 for the remaining period.
